(19) United States
(12) Patent Application Publication
(10) Pub. No.: US 2015/0166423 A1
(43) Pub. Date: Jun. 18, 2015

(54) Processes and Systems for In-Line HBR Oxidation and Cyclic Oxygen Shuttle

(71) Applicant: GTC Technology US, LLC, Houston, TX (US)

(72) Inventor: John J. Waycuilis, Cypress, TX (US)

(73) Assignee: GTC Technology US, LLC, Houston, TX (US)

(21) Appl. No.: 14/561,047

(22) Filed: Dec. 4, 2014

Related U.S. Application Data
(60) Provisional application No. 61/915,384, filed on Dec. 12, 2013.

(51) Int. Cl. C07C 1/30; B01J 19/24; C07C 17/10; C01B 7/09

(52) U.S. Cl. CPC C07C 1/30; C01B 7/096; B01J 19/245; C07C 17/10; C07C 2529/08; C07C 2529/40; B01J 2219/24

(57) ABSTRACT

An embodiment relates to a process for converting lower molecular weight, gaseous alkanes to higher hydrocarbons, such as olefins, higher molecular weight hydrocarbons, or mixtures thereof, that may use in-line hydrogen bromide oxidation for capture of hydrogen bromide produced in the process. An embodiment may provide a process for producing elemental halogen comprising: providing a first stream comprising a hydrogen halide; contacting the first stream with a metal oxide to form water, elemental halogen, and at least some metal halide, wherein the metal oxide comprises a metal capable of forming a plurality of stable oxidation states; and contacting the metal halide with an oxygen source to produce a regenerated metal oxide, wherein the oxygen source contacts the metal halide under conditions sufficient to avoid release of elemental halogen.

### CROSS-REFERENCE TO RELATED APPLICATIONS

The present application claims priority to U.S. Provisional Application No. 61/915,384, filed Dec. 12, 2013, the entire disclosure of which is incorporated herein by reference.

### BACKGROUND

Embodiments relate to a process for converting lower molecular weight, gaseous alkanes to higher molecular weight hydrocarbons that may be useful as fuels or chemical feedstocks. More particularly, embodiments relate to a process wherein a gas containing lower molecular weight alkanes is reacted with bromine vapor, to form alkyl bromides which in turn are reacted over a crystalline alumino-silicate catalyst to form paraffins, olefins, naphthenic and aromatic compounds or mixtures thereof and hydrobromic acid, and that may use in-line hydrogen bromide oxidation for capture of hydrogen bromide produced in the process and re-conversion to bromine.

Natural gas, which is primarily composed of methane and other light alkanes, has been discovered in large quantities throughout the world. Many of the locales in which natural gas has been discovered are far from populated regions which have significant gas pipeline infrastructure or market demand for natural gas. Due to the low density of natural gas, transportation thereof in gaseous form by pipeline or as compressed gas in vessels is expensive. Accordingly, practical and economic limits exist to the distance over which natural gas may be transported in gaseous form. Cryogenic liquefaction of natural gas (LNG) is often used to more economically transport natural gas over large distances. However, this LNG process is expensive and there are limited regasification facilities in only a few countries that are equipped to import LNG.

Another use of methane is as feed to processes for the production of methanol. Methanol is made commercially via conversion of methane to synthesis gas (CO and H₂) at high temperatures (approximately 1000° C.) followed by synthesis at high pressures (approximately 100 atmospheres). There are several types of technologies for the production of synthesis gas from methane. Among these are steam-methane reforming (SMR), partial oxidation (PDX), autothermal reforming (ATR), gas-heated reforming (GHR), and various combinations thereof. SMR and GHR operate at high pressures and temperatures, generally in excess of 600° C., and require expensive furnaces or reactors containing special heat and corrosion-resistant alloy tubes filled with expensive reforming catalyst. PDX and ATR processes operate at high pressures and even higher temperatures, generally in excess of 1000° C. As there are no known practical metals or alloys that can operate at these temperatures, complex and costly refractory-lined reactors and high-pressure waste-heat boilers to quench and cool the synthesis gas effluent are required. Also, significant capital cost and large amounts of power are required for compression of oxygen or air to these high-pressure processes. Thus, due to the high temperatures and pressures involved, synthesis gas technology is expensive, resulting in a high cost methanol product which limits higher-value uses thereof, such as for chemical feedstocks and solvents. Furthermore production of synthesis gas is thermodynamically and chemically inefficient, producing large excesses of waste heat and unwanted carbon dioxide, which tends to lower the conversion efficiency of the overall process. Fischer-Tropsch Gas-to-Liquids (GTL) technology can also be used to convert synthesis gas to heavier liquid hydrocarbons, however investment cost for this process is even higher. In each case, the production of synthesis gas represents a large fraction of the capital costs for these methane conversion processes.

Numerous alternatives to the conventional production of synthesis gas as a route to methanol or synthetic liquid hydrocarbons have been proposed. However, to date, none of these alternatives has attained commercial status for various reasons. Thus, a need exists for an economic process for the conversion of methane and other alkanes found in natural gas to olefins, higher molecular weight hydrocarbons including valuable aromatic compounds or mixtures thereof which, due to their higher density and value, are more economically transported thereby significantly aiding development of remote natural gas reserves. Further, a need exists for such a process that is relatively inexpensive, safe and simple.

### SUMMARY

An embodiment may provide a process for producing elemental halogen comprising: providing a first stream comprising a hydrogen halide; contacting the first stream with a metal oxide to form water, elemental halogen, and at least some metal halide, wherein the metal oxide comprises a metal capable of forming a plurality of stable oxidation states; and contacting the metal halide with an oxygen source to produce a regenerated metal oxide, wherein the oxygen source contacts the metal halide under conditions sufficient to avoid release of elemental halogen.

An embodiment may provide a process for producing higher hydrocarbons comprising: providing a first stream comprising lower molecular weight alkanes and a hydrogen halide; contacting the first stream with a metal oxide to form oxidation products comprising water, elemental halogen, and a metal halide; reacting at least a portion the methane and at least a portion of the elemental halogen to form halogenation products comprising alkyl halides and a first portion of produced hydrogen halide; and contacting at least a portion of the alkyl halides with a catalyst to produce synthesis products comprising higher hydrocarbons and a second portion of produced hydrogen halide.

An embodiment may comprise a system for producing higher hydrocarbons comprising: an oxidation unit comprising a metal oxide reactor bed for converting a hydrogen halide to a metal halide and elemental halogen, wherein at least a portion of the metal oxide in the metal oxide reactor bed is converted to a metal halide, and wherein the oxidation unit further comprises an offline metal oxide bed containing converted metal halide in fluid communication with an oxygen source; a bromination unit in fluid communication with the oxidation unit for reacting an alkane with the elemental halogen; and a synthesis unit in fluid communication with the bromination unit, wherein the synthesis unit comprises a catalyst for converting alkyl halides to higher hydrocarbons.

The features and advantages of the present invention will be readily apparent to those skilled in the art. While numerous changes may be made by those skilled in the art, such changes are within the spirit of the invention.

### BRIEF DESCRIPTION OF THE SEVERAL VIEWS OF THE DRAWINGS

These drawings illustrate certain aspects of some of the embodiments of the present invention, and should not be used to limit or define the invention.

FIG. 1 is a simplified block flow diagram of an embodiment of a process for producing higher hydrocarbons with inline hydrogen bromide oxidation.

FIGS. 2 to 5 are charts showing example of calculated equilibrium vapor-phase and solid-phase compositions from operation of a copper oxide solid-reactant bed

### DETAILED DESCRIPTION

Embodiments relate to a process for converting lower molecular weight, gaseous alkanes to higher molecular weight hydrocarbons that may be useful as fuels or chemical feedstocks. More particularly, embodiments relate to a process for converting lower molecular weight, gaseous alkanes to higher molecular weight hydrocarbons, such as paraffins, olefins, naphthenic and aromatic compounds, or mixtures thereof, that may use in-line hydrogen bromide oxidation for capture of hydrogen bromide produced in the process.

As utilized throughout this description, the term “lower molecular weight alkanes” refers to methane, ethane, propane, butane, pentane or any combination thereof including combinations such as natural gas. As used herein, the use of the term Cₙ refers to a hydrocarbon with a carbon number corresponding to the subscript “n”. In addition, the use of Cₙ+ refers to a hydrocarbon or a hydrocarbon group comprising hydrocarbons of “n” carbon atoms and/or any hydrocarbon having a number of carbon atoms greater than “n.” Therefore, the use of the designation C₅+ indicates that the fraction may include any proportion of C₅ hydrocarbons and may also include any hydrocarbons with 5 or more carbon atoms (e.g., C₆ hydrocarbons, C₇ hydrocarbons, etc.) As also utilized throughout this description, “alkyl halides” and “alkyl bromides” refer to mono, di, and tri halogenated and brominated alkanes, respectively.

Feed gas that may be used in the processes described herein may be from any suitable source, for example, any source of gas that provides lower molecular weight alkanes, whether naturally occurring or synthetically produced. Examples of sources of lower molecular weight alkanes for use in the processes of the present invention include, but are not limited to, natural gas, coal-bed methane, regasified liquefied natural gas, gas derived from gas hydrates and/or chlathrates, gas derived from anaerobic decomposition of organic matter or biomass, gas derived in the processing of tar sands, and synthetically produced natural gas or alkanes. Combinations of these may be suitable as well in some embodiments. Also, the feed gas may comprise natural gas which may be treated to remove sulfur compounds and carbon dioxide. In any event, it is important to note that embodiments may tolerate small amounts of carbon dioxide, e.g., less than about 2 mol %, in the feed gas.

As used herein, the term “higher hydrocarbons” when used in the context of a coupling, synthesis, or oligomerization reaction refers to higher molecular weight hydrocarbons having a greater number of carbon atoms than one or more hydrocarbon components of the feed gas, as well as olefinic hydrocarbons having the same or a greater number of carbon atoms as one or more hydrocarbon components of the feed gas stream. For instance, if the feed gas comprises natural gas, which may typically be a mixture of lower molecular weight alkanes, predominately methane, with lesser amounts of ethane, propane, and butane, and even smaller amounts of longer chain hydrocarbons such as pentane, hexane, etc., the “higher hydrocarbon(s)” produced according to the invention may include a C₂ or higher hydrocarbon, such as ethane, propane, butane, C₅+ hydrocarbons, aromatic hydrocarbons, etc., and optionally ethylene, propylene, and/or longer olefins.

While there are many advantages to the disclosed processes, only a few are discussed herein. Embodiments may allow for the capture of a hydrogen halide produced by the conversion of lower molecular weight alkanes to higher hydrocarbons according the process disclosed herein. The use of a metal oxide that may be converted to a metal halide with a plurality of stable oxidation states may allow for hydrogen halide to be captured while simultaneously producing elemental halogen. This process may allow oxygen to be introduced into the system without directly contacting a hydrocarbon containing stream. Further, complete separation of the hydrogen halide from the synthesis product stream may be avoided, allowing a more cost effective process to be implemented.

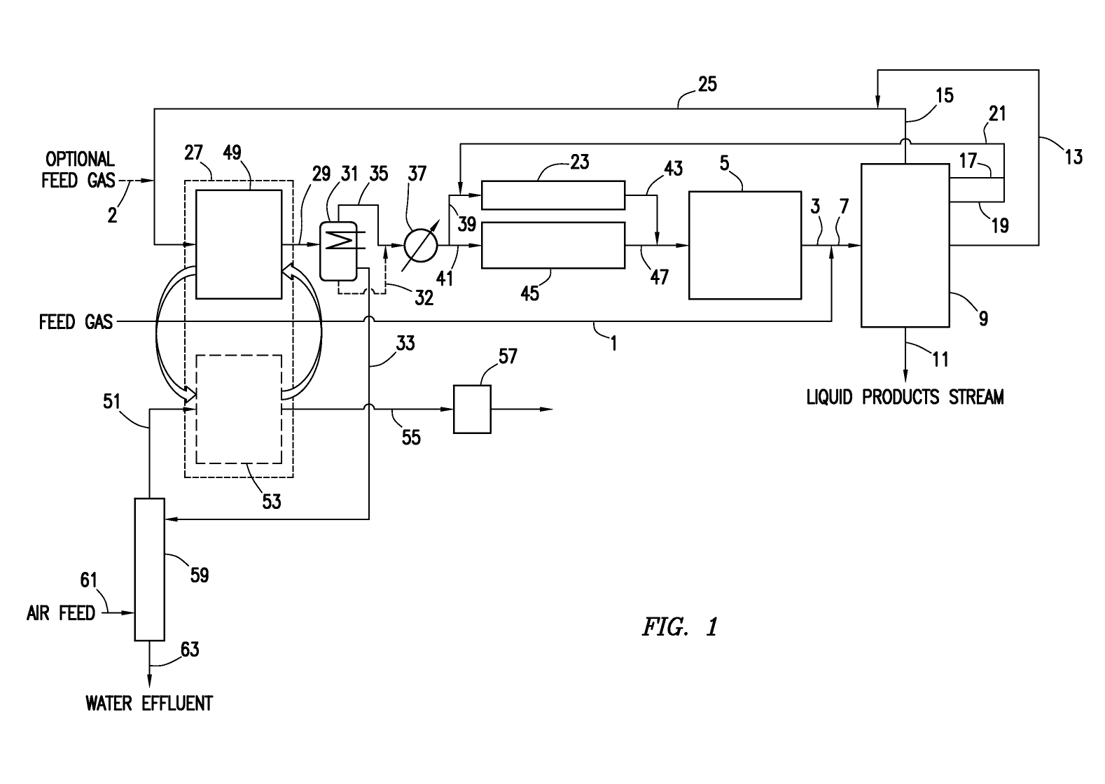

A block flow diagram generally depicting an embodiment of a process for producing higher hydrocarbons is illustrated in FIG. 1. A feed gas 1 may be combined with a synthesis product stream 3 from a synthesis unit 5. The synthesis product stream 3 may comprise methane, higher molecular weight hydrocarbons, hydrogen halide, and unreacted alkyl halides. The combined stream 7 may then pass to a separation unit 9 that may produce a liquid products stream 11, a hydrogen halide stream 13, a methane stream 15, a C₂ stream 17, and a C₃ stream 19. The liquid products stream 11 may comprise C₄ and higher hydrocarbons. The methane stream 15 may comprise mostly methane, but which may also contain some ethane and hydrogen halide. The C₂ stream 17 may comprise mostly C₂ hydrocarbons but which may also contain some hydrogen halide. The C₃ stream 44 may comprise mostly C₃ hydrocarbons but which may also contain some hydrogen halide. The hydrogen halide stream 13 may comprise mostly hydrogen halide, but which may also contain some C₂ and C₃ hydrocarbons. The C₂ stream 17 and C₃ stream 19 may be combined to form C₂-C₃ recycle stream 21, for recycle to a C₂₊ halogenation unit 23. The hydrogen halide stream 13 may be combined with methane stream 15 to form C₁ recycle stream 25. In the embodiment illustrated in FIG. 1, an optional feed gas 2 containing high-purity methane may optionally be introduced into the C₁ recycle stream 25. The C₁ recycle stream 25 may then be supplied to an oxidation unit 27 where the hydrogen halide in the C₁ recycle stream 25 may be converted to molecular halogen and water vapor or steam, as described in more detail herein. Representative halogens include bromine (Br₂) and chlorine (Cl₂). While the description herein uses bromine as an exemplary embodiment, it is also anticipated that chlorine may be used although not necessarily with identical conditions or results.

The methane in the C₁ recycle stream 25 may pass through the oxidation unit 27 along with the elemental halogen and water vapor or steam produced in the oxidation unit 27 to form an oxidation product stream 29. The oxidation product stream 29 may then pass to the condenser 31 which may be operated at a temperature that is below the dewpoint of water and above the dewpoint of the halogen as would be apparent to the skilled practitioner, so as to remove a substantial amount of the water vapor contained in oxidation product stream 29, forming a water condensate stream 33 comprising mostly water but that may also contain some halogen. The vapor effluent 35 exiting condenser 31 may then be reheated in in-line heater 37 to a temperature in the range of about 150° C. to about 300° C. The reheated vapor effluent stream exiting in-line heater 37 may then be divided into two portions, a first portion stream 39 and a second portion stream 41, which may be supplied to the C₂₊ halogenation unit 23 and the C₁₊ halogenation unit 41. Within the C₂₊ halogenation unit 23, ethane, ethylene, propane, propylene and any other heavier hydrocarbons contained in C₂-C₃ recycle stream 21 may react with the elemental halogen contained in first portion stream 39 to form C₂₊ alkyl halide stream 43. The C₂₊ alkyl halide stream 43 may comprise C₂ and higher alkyl halides, hydrogen halide, and unreacted C₂ and higher hydrocarbons. The balance of reheated vapor effluent leaving in-line heater 37 comprises the second portion stream 41, which may be routed to C₁₊ halogenation unit 45. Within the C₁₊ halogenation unit 41, the methane and small amounts of C₂ and heavier hydrocarbons may react with the elemental halogen contained in second portion stream 41 to form C₁₊ alkyl halide stream 47. The C₁₊ alkyl halide stream 47 may comprise mostly C₁ alkyl halides and small amounts of C₂ and heavier alkyl halides, hydrogen halide and unreacted methane. The C₂₊ alkyl halide stream 43 may be combined with C₁₊ alkyl halide stream 47 which may then pass to the synthesis unit 5 wherein the alkyl halides may react over a suitable catalyst to form the synthesis product stream 3, wherein the whole process may be repeated. Accordingly, the process illustrated in FIG. 1 may be used to produce a liquid hydrocarbon product comprising C₄₊ hydrocarbons (e.g., liquid products stream 11) from lower molecular-weight hydrocarbons, such as methane, ethane, propane, etc. (e.g., feed gas 1).

As generally described above in reference to FIG. 1, the feed gas 1 comprising lower molecular weight alkanes may be combined with a synthesis product stream 3 before being sent to a separation unit 9. The feed gas 1, the synthesis product stream 3, and the resulting combined stream 7 may be at a pressure ranging, for example, from about 1 bar to about 50 bar. The separation unit 9 may comprise any process or unit capable of separating hydrocarbons into fractions and also separating a hydrogen halide fraction. As used herein, a “fraction” refers to a separation product stream that is predominantly a stated species, but may include other species that enter the separation process. For example a C₁ fraction may comprise about 98 mol % methane but may also include up to about 2 mol % C₂ and traces of C₃ hydrocarbons. Suitable processes are known to those skilled in the art and may include processes such as: refrigerated condensation and distillation or extractive distillation, cryogenic expander processes combined with distillation or extractive distillation, pressure swing absorption units, and solvent based separation systems including circulating solvent systems. Further, two separate processes may be operated in series within separation unit 9 such as a first process to separate hydrogen halide from hydrocarbons and a second process to subsequently fractionate the methane and higher molecular components into C₁, C₂-C₃, and C₄₊ fractions. Such processes may be used to produce a C₄ and higher hydrocarbon fraction (e.g., liquid products stream 11), a C₂-C₃ hydrocarbon fraction (e.g., a C₂ stream 17, a C₃ stream 19, or a combined C₂-C₃ hydrocarbon fraction), a C₁ hydrocarbon fraction (e.g., methane stream 15) that may also include a small amount, for example, less than about 2 mol % of C₂, and a hydrogen halide fraction (e.g., hydrogen halide stream 13) containing the hydrogen halide produced in the C₁₊ halogenation unit 45, C₂₊ halogenation unit 23 and synthesis unit 5, for example. The C₂ stream 17 and C₃ stream 19 may be combined to form C₂-C₃ recycle stream 21 that may be recycled to the C₂₊ halogenation unit 23. Alternatively, the C₂ stream 17 and C₃ stream 19 may be separately recycled. The C₄ and higher hydrocarbon fraction may leave the separation unit 9 and the process as liquid products stream 11.

In an alternate embodiment the C₂-C₃ hydrocarbon fraction may be withdrawn as additional products from the process rather than recycled, obviating the need for C₂₊ halogenation unit 23.

In another embodiment, if the feed gas 1 comprises essentially pure methane, the C₁ hydrocarbon fraction may include the relatively small amount of C₂ hydrocarbon produced in the synthesis unit 5, for example, comprising less than 2 mol % of C₂ of the fraction, and also include the hydrogen halide entering the separation unit 9 and may leave the separation unit 9 in methane stream 15; in this case C₂-C₃ recycle stream 21 may comprise mostly C₃ hydrocarbons and may include only small amounts of C₂ hydrocarbons and hydrogen halide.

The amount of C₂-C₃ hydrocarbons in the methane stream 15 may be varied by changing the conditions in the separation unit 9 but, in some embodiments, may be maintained at low concentration levels of less than about 2 mol % C₂ of the fraction, and less than about 0.1 mol % C₃ of the fraction. As shown in FIG. 1, the methane stream 15 may comprise the methane in feed gas 1, unreacted methane and only small amounts of light hydrocarbons (e.g., less than about 2 mol % C₂ and less than about 0.1 mol % C₃) not recovered in separation unit 9. Because of the higher reactivity of ethane with halogen (e.g., bromine) and the even higher reactivity of propane with halogen (e.g., bromine) relative to the reactivity of methane with halogen, it has been discovered that if C₂, C₃ or both are present in the methane stream 15 in concentrations above these limits then the ethane and propane become highly polybrominated and lead to the production of soot at much lower temperatures and well before appreciable reaction of the methane with halogen occurs. However, it has also been discovered that ethane and propane can be brominated and poly-brominated separately without significant soot formation as long as an excess of these C₂ and C₃ alkanes is present, e.g., such that the bromine to alkane molar ratio is less than about 0.75:1, and may also be greater than about 0.5:1.

As illustrated in FIG. 1, the methane stream 15 may be combined with hydrogen halide stream 13 to form C₁ recycle stream 25 that may then be passed to the oxidation unit 27 to allow for conversion of the hydrogen halide into elemental halogen. Alternatively, the hydrogen halide stream 13 and methane stream 15 may be separately recycled. In an embodiment, the oxidation unit 27 may be capable of generating elemental halide without the need to contact the hydrogen halide directly with elemental oxygen. In this embodiment, a metal oxide may be used to react with a hydrogen halide contained in a process stream. For example, the oxidation unit 27 may contain a metal oxide reactor bed 49, which may be used to capture and oxidize the hydrogen halide in the C₁ recycle stream 25. The metal of the metal oxide may be selected from any metal that forms a plurality of stable oxidation states. For example, the metal of the metal oxide may include chromium (Cr), iron (Fe), copper (Cu), tin (Sn), or vanadium (V) or mixtures thereof. The metal(s) may be selected based on such considerations as the impact of its physical and thermodynamic properties relative to the desired operating temperature, cost and also any potential environmental and health impacts. Copper or iron are may be used in some embodiments, because these metals have the property of not only forming oxides but halide salts with multiple oxidation states as well, within in a practical temperature range of less than about 350° C. In some embodiments, copper may be employed as the metal in the metal oxide. The metal oxide reactor bed 49 may comprise a solid metal oxide in particle form, or as an active layer of solid metal oxide dispersed on a suitable inert attrition-resistant support, for example a silica, alumina, titania, zirconia, etc. An alumina support with a specific surface area between about 50 m²/g to about 200 m²/g may be used in some embodiments.

Without wishing to be limited by theory, it is believed that when a gaseous stream (e.g., C₁ recycle stream 25) comprising a hydrogen halide (e.g., hydrogen bromide) contacts a metal oxide (e.g., metal oxide reactor bed 49) with a plurality of oxidation states, the hydrogen halide may be removed from the stream with the production of the corresponding molecular halogen. The rate of reaction and the production of the corresponding elemental halogen may depend on the operating conditions, and is particularly dependent on the temperature at which the reaction occurs. In an embodiment, the capture of a hydrogen halide with a metal oxide/metal halide with multiple oxidation states may occur at a temperature ranging from about 150° C. to about 350° C. and more alternatively at a temperature within a range about 200° C. to about 300° C. The reactions may be exemplified by the following equations using copper oxide and hydrogen bromide as examples:

2 HBr(g)+CuO(s)→CuBr₂(s)+H₂O(g) (K_{eq} on the order of 7.8×10⁸ @ 250° C.)

2 CuBr₂(s)→2 CuBr(s)+Br₂(g) (K_{eq} on the order of 3×10⁻¹ @ 250° C.)

Upon contact of the hydrogen halide with the metal oxide, the reaction may continue until a large portion of the metal oxide on the metal oxide reactor bed 49 may be converted into a metal halide. However, before all, or substantially all, of the metal oxide has been converted to metal halide, the metal oxide reactor bed 49 may be taken offline and regenerated. As illustrated on FIG. 1, an oxygen source 51 may be supplied to offline metal oxide reactor bed 53. A large portion of the metal oxide in offline metal oxide reactor bed 53 may have been converted to metal halide by reaction with the hydrogen halide in C₁ recycle stream 25, for example. The oxygen source 51 may be delivered to the offline metal oxide reactor bed 53 via a blower or compressor at a pressure in the range of about ambient to about 20 bar. The oxygen source 51 may be preheated to a temperature in the range of about 25° C. to about 300° C. depending on the desired reaction temperature. The reaction products may be controlled during regeneration of offline metal oxide reactor bed 53 through manipulation of the reaction conditions, specifically the temperature at which the reaction is allowed to occur and the flow rate of the oxygen source. In an embodiment, the oxidation of an iron oxide/iron halide with multiple oxidation states may occur at a temperature ranging from about 25° C. to about 100° C. and alternatively at a temperature ranging about 30° C. to about 70° C. In another embodiment, the oxidation of copper oxide/copper halide may occur at temperatures up to about 150° C. and alternatively at a temperature in the range of about 50° C. to about 100° C. If the oxidation occurs at a temperature at which the reversible equilibrium favors the higher-oxidation-state metal halide, little to no molecular halogen may be produced during oxidation. The reactions may be exemplified by the following equations using copper bromide as an example:

2 CuBr(s)+O₂(g)→2 CuO(s)+Br₂(g) (K_{eq} on the order of 2.6×10⁶ @ 100° C.)

2 CuBr(s)+Br₂(g)→2 CuBr₂(s) (K_{eq} on the order of 2.7×10⁴ @ 100° C.)

Based on these assumed reaction mechanisms, a metal halide with multiple oxidation states may be used to shuttle elemental oxygen from an oxygen source 51 into the process for producing higher hydrocarbons without directly contacting a hydrocarbon stream (e.g., C₁ recycle stream 25) with elemental oxygen. The oxygen source 51 may include, but is not limited to, air, oxygen enriched air, pure oxygen, any other source of elemental oxygen, or any combination thereof. Further, the halogen used in the process may be contained in a metal halide salt and be retained within the process. Other metal oxide/metal halide systems in which the metal may exist in multiple oxidation states, such as chromium (Cr), tin (Sn) or Vandium (V), can in principle also be utilized except that these metal halides are more stable to decomposition, requiring substantially higher temperatures before significant elemental halogen is evolved. However, without wishing to be limited by theory, some metal halides may be directly reactive with alkanes to yield alkyl halides without free elemental halogen being formed. Further, mixtures of one or more metal halides, for example copper bromide and vanadium bromide, or iron bromide and copper bromide may be employed in certain embodiments to implement the invention. Regeneration off-gas 55 comprising mostly inert components contained in the oxygen source (e.g. nitrogen from air) and excess oxygen and smaller amounts other gases from the regeneration may be withdrawn from the offline metal oxide reactor bed 53. The regeneration off-gas 55 may then be treated in scrubbing unit 57, for example, to remove any trace amounts of halogen that might be normally present, or larger amounts of halogen that might be intermittently present due to process upsets or mechanical failures, etc., and then vented to the atmosphere or utilized in another process.

The cyclic nature of this process may require a plurality of reactor vessels to allow at least one metal oxide reactor bed (e.g., metal oxide reactor bed 49) to contact a stream containing a hydrogen halide while another reactor (offline metal oxide reactor bed 53) containing a metal halide may be allowed to contact an oxygen source (e.g., oxygen source 51). Multiple metal oxide/metal halide reactor beds may be used with any number of reactors cycling between oxidation and regeneration to allow for the system to be run continuously. Such reactors may comprise fixed bed reactors, radial bed reactors, fixed fluidized bed reactors (e.g., a fluidized bed that remains substantially confined within a single vessel), or any other suitable reactor type. Alternatively, a circulating fluidized bed reactor or moving bed reactor design may be used. In this embodiment, the metal oxide may contact a stream containing a hydrogen halide in one reactor or reaction zone and then be transported to another reactor or reaction zone to contact an oxygen source. Exemplary moving bed reactors may include vertical moving bed reactors, radial moving bed reactors, circulating fluidized bed reactors (e.g., with multiple reactors or reaction zones), or any other reactor configuration allowing for physical transport of a solid reactant or catalyst between reaction zones.

As shown in FIG. 1, the oxidation unit 27 may result in an aqueous condensate stream 33 as a result of water generation during the hydrogen halide capture reaction. The aqueous condensate stream 33 may be formed by routing the effluent stream 29 leaving the oxidation unit 27 through a condenser 31 maintained at a temperature below the dew point of water, but above the dew-point of the elemental halogen (e.g., bromine) in the vapor mixture at the selected operating pressure. As illustrated, the effluent stream 29 may leave metal oxide reactor bed 49 and may then be supplied to condenser 31. The condenser 31 may produce an aqueous phase containing mostly water but also some small amount of halogen that may be limited by the solubility of halogen in water. The aqueous phase may leave the condenser 31 as aqueous condensate stream 33. In an alternative embodiment, the condenser 31 may be maintained at a low temperature to maximize the condensation of both water and halogen; which may then be physically separated by decanting the less dense aqueous phase, thereby removing most of the water into aqueous condensate stream 33. Liquid halogen 32 may also be separated in condenser as the liquid halogen 32 may be denser and have limited solubility for water. The liquid halogen 32 may be recombined with vapor effluent 35, and then heated and vaporized in in-line heat exchanger 37.

The resulting aqueous condensate stream 33 may be passed through a stripping vessel 59, for example, to remove any elemental halogen, hydrocarbons, and possibly any hydrogen halide absorbed in the aqueous phase. The stripping vessel 59 may use a stripping gas 61, such as air, along with suitable process conditions so that any elemental halogen absorbed in the aqueous phase may be stripped into the vapor phase and carried along with the stripping gas 61, which may leave the stripping vessel 59 as oxygen source 51. In an embodiment, the oxygen source 51 may then pass to the offline metal oxide bed 53 undergoing regeneration. Such an arrangement may allow for any elemental halogen to be recaptured by the metal halide for further use in the system. The water effluent 63 exiting the stripping vessel 59 may contain only trace amounts of a halogen and may be treated in an additional process to remove any traces of halogen and halides in the stream for disposal, or may be used in other processes requiring an aqueous stream.

With continued reference to FIG. 1, the oxidation product stream 29 leaving the oxidation unit 27 may comprise any light hydrocarbons that entered the oxidation unit and elemental halogen. The light hydrocarbons in the oxidation product stream 29 may comprise any methane and any higher hydrocarbons contained in the C₁ recycle stream 25. Some trace amounts of hydrogen halide may pass through the oxidation unit 27 under some operating conditions, therefore the operating parameters may be controlled to minimize the amount of hydrogen halide which may pass unreacted through oxidation unit 27, since any hydrogen halide present would become dissolved in the aqueous condensate 33 and ionize forming hydrohalic acid which would pass through stripping vessel 59 and be retained in water effluent 63 and hence represent a loss of halogen from the process. The hydrocarbons and elemental halogen contained in oxidation product stream 29 may then pass through condenser 31 and in-line heater 37 to C₁₊ halogenation unit 45 and C₂₊ halogenation unit 23. The C₁₊ and C₂₊ halogenation units 45 and 21 may comprise one or more reactors. The C₂-C₃ recycle stream 21 may also pass to the C₂₊ halogenation unit 23.

As illustrated on FIG. 1, the second portion stream 41 that may comprise methane and elemental halogen may be fed to the C₁₊ halogenation unit 45. The mixture of methane and elemental halogen vapor fed to the C₁₊ halogenation unit 45 may have a molar ratio of methane to elemental halogen vapor in excess of 2.5:1. The flow rate of the feed gas 1 may be varied to ensure that the molar flow rate of methane in C1 recycle stream 25 is such that the molar ratio of methane to elemental halogen vapor exceeds 2.5:1 in the second portion stream 41. Further, the C₂-C₃ recycle rate of the C₂-C₃ recycle stream 21 may be adjusted to ensure that the ratio of higher alkanes to halogen is in excess of about 1.33:1 in the feed to the C₂₊ halogenation unit 45. The C₁₊ halogenation unit 45 may have an inlet pre-heater zone for heating the mixture to a reaction initiation temperature in the range of about 250° C. to about 400° C. such that the C₁ alkyl halide stream 47 may reach a temperature in the range of about 530° C. to about 570° C. In the C1+ halogenation unit 45, the methane and lower molecular weight alkanes may react exothermically with halogen vapor at a relatively low temperature in the range of about 250° C. to about 600° C., and at a pressure in the range of about 1 bar to about 40 bar to produce gaseous alkyl halides and hydrogen halide. In specific embodiments, the C1+ halogenation unit 45 may be operated at a temperature in the range of about 450° C. to 570° C. The upper limit of the operating temperature range may be greater than the upper limit of the reaction initiation temperature range to which the feed mixture is heated due to the exothermic nature of the bromination reaction. In the case of methane reacting with bromine, the formation of methyl bromide may occur in accordance with the following general reaction:

CH₄(g)+Br₂(g)→CH₃Br(g)+HBr(g)

This reaction may occur with a significantly high degree of selectivity to methyl bromide. For example, in the case of bromine reacting with a molar excess of methane at a methane to bromine ratio of 3:1, a temperature of about 500° C. and a residence time of about 60 seconds, about 90% selectivity to the mono-halogenated methyl bromide may occur. Small amounts of dibromomethane and trace amounts of tribromomethane may also form in the bromination reaction. Small amounts of higher alkanes, such as ethane, propane contained in the C1 recycle stream 25, may also be readily brominated resulting in mono and multiple brominated species such as ethyl bromides and propyl bromides. If a methane to bromine ratio of significantly less than 2.5 to 1 is utilized, substantially lower selectivity to methyl bromide may occur and significant formation of undesirable carbon soot may be observed. Further, the amount of water vapor contained in the feed into the C₁₊ halogenation unit 45 should be minimized. It has been discovered that minimization of water vapor in the feed to the C₁₊ halogenation unit 45 may minimize the formation of unwanted carbon dioxide thereby increasing the selectivity of alkane bromination to alkyl bromides and eliminating the large amount of waste heat generated in the formation of carbon dioxide from alkanes.

In the C₂₊ halogenation unit 23, the C₂₊ hydrocarbons contained in the C₂-C₃ recycle stream 21 may react exothermically with halogen vapor from the first portion stream 39 at a relatively low temperature, for example, in the range of about 250° C. to about 400° C., and at a pressure in the range of about 1 bar to about 40 bar to produce gaseous higher alkyl halides and hydrogen halide. In particular embodiments, the C₂₊ halogenation unit 23 may be operated at a temperature in the range of 300° C. to about 375° C. In some embodiments, the C2₊ halogenation unit 23 may have an inlet pre-heater zone for heating its feed to a reaction initiation temperature in the range of about 200° C. to about 350° C. The C₂ alkyl halide stream 43 may reach a temperature in the range of about 350° C. to about 375° C. As previously described, halogen may be supplied to C₂₊ halogenation unit 23 by way of first portion stream 39. The portion of reheated vapor effluent stream from in-line heater 37 going to first portion stream 39 may be adjusted such that the molar ratio of halogen to the sum of C₂ plus C₃ hydrocarbons in C₂-C₃ recycle stream 21 may be in the range of about 0.75 to 0.5 in the combined feed to C₂₊ halogenation unit 23. While FIG. 1 illustrates a separate C₂₊ halogenation unit 23 and C₁₊ halogenation unit 45, it should be understood that embodiments may include a combined C₁₊ and C₂₊ halogenation unit or multiple different halogenations units, for example, further comprising as separate C3₊ halogenation unit.

As will be appreciated by those of ordinary skill in the art, with the benefit of this disclosure, the reactions in C₁₊ halogenation unit 45 and in C₂₊ halogenation unit 23 may be a homogeneous gas phase reaction or a heterogeneous (catalytic) reaction. Examples of suitable catalysts that may be utilized in C₁₊ halogenation unit 45 or C₂₊ halogenation unit 23 may include, but are not limited to, platinum, palladium, or supported non-stoichiometric metal oxy-halides such as FeOₓBr_{y} or FeOₓCl_{y} or supported stoichiometric metal oxy-halides such as TaOF₃, NbOF₃, ZrOF₂, SbOF₃ as described in Olah, et al, J. Am. Chem. Soc. 1985, 107, 7097-7105, which is incorporated herein in its entirety. In some embodiments, a multi-zone halogenation reactor may be used in the C₁₊ halogenation unit 45. In this embodiment, the vapor effluent stream 35 comprising halogen and alkanes may be heated to a temperature sufficient to initiate thermal bromination of the alkanes. A downstream zone may then comprise one or more catalytic materials to complete the halogenation reaction and increase the selectivity of the reaction products to mono-halogenated methane (e.g., methyl bromide). The C₂-C₃ recycle stream 21 may be recycled into a downstream zone of the C₁₊ halogenation unit 45 in some embodiments, or, as illustrated, fed to a separate, parallel C₂₊ halogenation unit 23 reactor.

As shown in FIG. 1, the C₁ alkyl halide stream 47 leaving the C₁₊ halogenation unit 45 and comprising alkyl halides (e.g., alkyl bromide) and hydrogen halide may be combined with the C2 alkyl halide stream 43 and passed to the synthesis unit 5. In alternative embodiments, the C₁ alkyl halide stream 47 and C₂ alkyl halide stream 43 may be separately fed to the synthesis unit 5. The C₁ alkyl halide stream 47 leaving the C₁₊ halogenation unit 45 may comprise alkyl halides, hydrogen halide, and any unreacted light hydrocarbons. The C₁ alkyl halide stream 47 may be withdrawn from the C₁₊ halogenation unit 45 and partially cooled in a heat exchanger before flowing to the synthesis unit 5. The temperature to which the C₁ alkyl halide stream 47 is partially cooled in the heat exchanger may range from, for example, about 150° C. to about 350° C. when it is desired to convert the alkyl halides to higher molecular weight hydrocarbons in the synthesis unit 5, or to range of about 150° C. to about 450° C. when it is desired to convert the alkyl halides to olefins in the synthesis unit 5. In the synthesis unit 5, the alkyl halides may react exothermically in the presence of a catalyst. Non-limiting examples of suitable catalysts for use in synthesis unit 5 include crystalline alumino-silicate zeolites, partially de-aluminated crystalline alumino-silicate zeolites which may be partially amorphous in character, or ion-exchanged zeolites. The temperature and pressure employed in the synthesis unit 5, as well as the zeolite catalyst, may determine the products that are formed from the reaction of alkyl halides occurring in the synthesis unit 5.

In an embodiment, the crystalline alumino-silicate catalyst employed in the synthesis unit 5 may be a zeolite catalyst, such as a ZSM-5 zeolite catalyst, when it is desired to form higher molecular weight hydrocarbons. The zeolite catalyst may be used in the hydrogen, sodium, magnesium form, or any combination thereof. The zeolite may also be modified by ion exchange with other alkali metal cations, such as Li, Na, K or Cs, with alkali-earth metal cations, such as Mg, Ca, Sr or Ba, or with transition metal cations, such as Ni, Mn, V, W, or to the hydrogen form. Other zeolite catalysts having varying pore sizes and acidities, which are synthesized by varying the alumina-to-silica ratio or that have been modified by chemical de-alumination may be used in the synthesis reactor as will be evident to a skilled artisan.

When it is desired to form olefins from the reaction of alkyl halides in the synthesis unit 5, the crystalline alumino-silicate catalyst employed in the synthesis reactor may be a zeolite catalyst. For example, the zeolite catalyst may be an X type, Y type or SAPO zeolite catalyst, although other zeolites with differing pore sizes and acidities, which are synthesized by varying the alumina-to-silica ratio may be used in certain embodiments as will be evident to a skilled artisan. The zeolite catalyst may be used in a protonic form, a sodium form, or a mixed protonic/sodium form. The zeolite catalyst may also be modified by ion exchange with other alkali metal cations, such as Li, K or Cs, with alkali-earth metal cations, such as Mg, Ca, Sr or Ba, or with transition metal cations, such as Ni, Mn, V, W, or to the hydrogen form. These various alternative cations may have an effect of shifting reaction selectivity. Other zeolite catalysts having varying pore sizes and acidities, which are synthesized by varying the alumina-to-silica ratio may be used in the synthesis reactor as will be evident to a skilled artisan.

The temperature at which the synthesis unit 5 is operated may be an important parameter in determining the selectivity of the reaction to higher molecular hydrocarbons or to olefins. Where a catalyst is selected to form higher molecular weight hydrocarbons in the synthesis unit 5, the synthesis unit 5 may be operated at a temperature ranging from about 150° C. to about 450° C. At the low end of the temperature range, with methyl bromide reacting over ZSM-5 zeolite at temperatures of about 150° C., low methyl bromide conversion on the order of 20% is noted, but with a high selectivity towards C₅+ products. At increasing temperatures approaching 400° C., methyl bromide conversion increases towards 95% or greater, however selectivity towards C₅+ products decreases slightly and selectivity towards lighter products increases. At temperatures approaching about 450° C., almost complete conversion of methyl bromide may occur but may result in further increased yields of light hydrocarbons such as undesirable methane and may significantly increase the rate of coke formation. Notably, in the case of the alkyl bromide reaction over the zeolite ZSM-5 catalyst, cyclization reactions may also occur such that the C₇+ fractions are composed primarily of substituted aromatics. Surprisingly, very little ethane or C₂-C₃ olefin components may be formed. In the optimum operating temperature range of between about 350° C. and about 400° C., as a byproduct of the reaction, a small amount of carbon may build up on the catalyst over time during operation, causing a decline in catalyst activity over a range of hours, up to about 24 hours, depending on space velocity, the reaction conditions and the composition of the feed gas. Without wishing to be limited by theory, it is believed that higher reaction temperatures above about 400° C., associated with the formation of methane may favor the thermal cracking of alkyl bromides and formation of carbon or coke and hence an increase in the rate of deactivation of the catalyst. Conversely, temperatures at the lower end of the range, particularly below about 300° C. may also contribute to coking due to a reduced rate of desorption of heavier products from the catalyst. Hence, operating temperatures in the range of about 350° C. to about 400° C. in the synthesis reactor may balance increased selectivity of the desired C₅+ products and lower rates of deactivation due to carbon formation, against higher conversion per pass, which may minimize the quantity of catalyst, recycle rates and equipment size required.

Where a catalyst is selected to form olefins in the synthesis unit 5, the synthesis unit 5 may be operated at a temperature ranging from about 250° C. to about 500° C. Temperatures above about 450° C. in the synthesis unit 5 may result in increased yields of light hydrocarbons, such as undesirable methane and also deposition of coke, whereas lower temperatures increase yields of ethylene, propylene, butylene and heavier molecular weight hydrocarbon products. Notably, in the case of the alkyl bromide reaction over a 10× zeolite catalyst, it is believed that cyclization reactions may also occur such that the C₇+ fractions contain substantial substituted aromatics. At increasing temperatures approaching 400° C., it is believed that methyl bromide conversion increases towards 90% or greater, however selectivity towards C₅+ products may decrease and selectivity towards lighter products, particularly olefins may increase. At temperatures exceeding 550° C., it is believed that a high conversion of methyl bromide to methane and carbonaceous coke may occur. In an operating temperature range of between about 300° C. and about 450° C., as a byproduct of the reaction, a lesser amount of coke may build up on the catalyst over time during operation, causing a decline in catalyst activity over a range of hours, depending on the space velocity, reaction conditions and the composition of the feed gas. Without wishing to be limited by theory, it is believed that reaction temperatures above about 400° C., associated with the formation of methane, favor the thermal cracking of alkyl bromides and formation of carbon or coke and hence an increase in the rate of deactivation of the catalyst. Conversely, temperatures at the lower end of the range, particularly below about 300° C. may also contribute to coking due to a reduced rate of desorption of heavier products from the catalyst. Hence, operating temperatures within the range of about 250° C. to about 500° C., and alternatively in the range of about 300° C. to about 450° C., in the synthesis unit 5 balance increased selectivity of the desired olefins and C₅+ products and lower rates of deactivation due to carbon formation, against higher conversion per pass, which minimizes the quantity of catalyst, recycle rates, and equipment size required.

The catalyst may be periodically regenerated in situ, by isolating the synthesis unit 5 from the normal process flow, purging with an inert gas at a pressure in the range of about 1 bar to about 40 bar and an elevated temperature in the range of about 400° C. to about 650° C. to remove unreacted material adsorbed on the catalyst insofar as is practical, and then subsequently oxidizing the deposited carbon to CO₂ by addition of air or inert gas-diluted oxygen to synthesis reactor at a pressure in the range of about 1 bar to about 40 bar and an elevated temperature in the range of about 400° C. to about 650° C. Carbon dioxide and residual air or inert gases are vented from the synthesis reactor during the regeneration period. The resulting gases may be diverted to the oxidation unit 27 in order to recapture any halogens released during the regeneration cycle, or the gases may be directed to a clean-up unit for removal of any trace halogens prior to being vented to the atmosphere.

The synthesis product stream 3 from the synthesis reactor 5, which may comprise methane, hydrogen halide and higher molecular weight hydrocarbons, olefins or mixtures thereof, may be withdrawn from the synthesis unit 5 and cooled to a temperature in the range of about 100° C. to about 500° C. using any suitable heat exchange device or quenching technique. The synthesis product stream 3 may then be combined with the feed gas 1 and the process may be repeated so as to operate in a continuous fashion.

To facilitate a better understanding of the present invention, the following examples of certain aspects of some embodiments are given. In no way should the following examples be read to limit, or define, the scope of the invention.

### EXAMPLE 1

A laboratory test setup was used to demonstrate the process described herein. This example was used to demonstrate oxidation of hydrogen bromide with a metal oxide. A mixture of simulated process recycle gas is composed of about 54 mol % methane, about 26 mol % hydrogen bromide gas, and about 20% nitrogen to act as an inert tie-element for calculating a mole percent is fed to a tubular reactor containing approximately 20 cm³ of a “solid reactant” composed of iron oxide dispersed on a porous inert support. The solid reactant contains approximately 20 wt % (as Fe) iron oxide dispersed on 3/16th inch spherical porous beads of gamma-alumina with a specific surface area of approximately 30 m²/g, manufactured by St. Gobain. The externally-insulated reactor tube is maintained at an external wall temperature in the range of approximately 150° C. to 200° C. by electrical resistance heating tape under the insulation to prevent excessive heat loss. Evolution of elemental bromine is observed in the reactor effluent, demonstrating that hydrogen bromide can be oxidized by iron oxide to yield elemental bromine.

### EXAMPLE 2

This example was used to demonstrate regeneration of a metal bromide with an oxygen containing gas mixture. Fifty (50) g of FeBr3 is dissolved in minimal water and imbibed into ˜75 cm3 of St. Gobain spherical alumina support ( 3/16th spheres). This material is loaded into a reactor tube composed of glass-lined steel pipe that is electrically heat-traced and insulated. N₂ gas was flowed at a rate at ˜15 ml/min though the reactor while ramping up heating of the reactor tube to 150° C. After a period of time bromine evolution is observed in the effluent gas. N₂ gas flow is continued until Br₂ evolution fell off significantly, then heating is reduced to cool reactor tube to ˜50° C. under continued N₂. Due to the heating under N₂ flow FeBr₃ is reduced to FeBr₂ according to the following reversible reaction:

2FeBr₃(s)=2FeBr₂(s)+Br₂(g)

With temperature at ˜50° C., a mixture of N₂ and O₂ at total flow rate of about 15 ml/min is initiated. Periodic samples of the inlet and outlet O₂ content are taken as a function of time and recorded. Table 1 below shows that FeBr₂ at 50° C. can absorb elemental oxygen from a nitrogen/oxygen mixture according to the following reaction:

6FeBr₂(s)+3/2O₂(g)=Fe₂O₃+4FeBr₃

TABLE 1

| Time | Inlet O₂ Conc. | Outlet O₂ Conc. |
|---|---|---|
| (minutes) | (mol %) | (mol %) |
| 30 | 15.03 | 5.06 |
| 90 | 18.17 | 3.50 |
| 120 | 18.53 | 8.90 |
| 150 | 18.53 | 14.32 |

### EXAMPLE 3

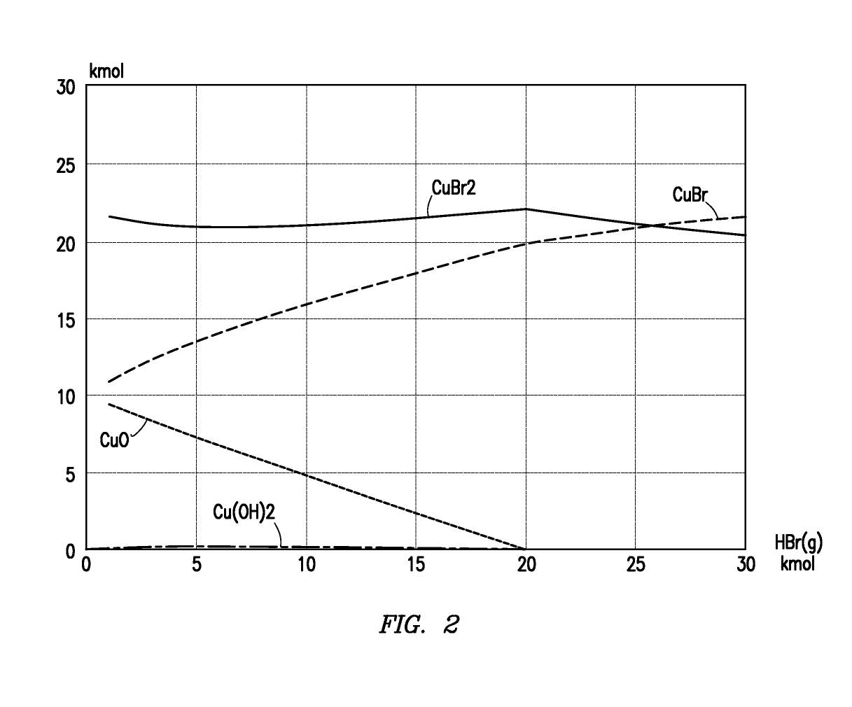

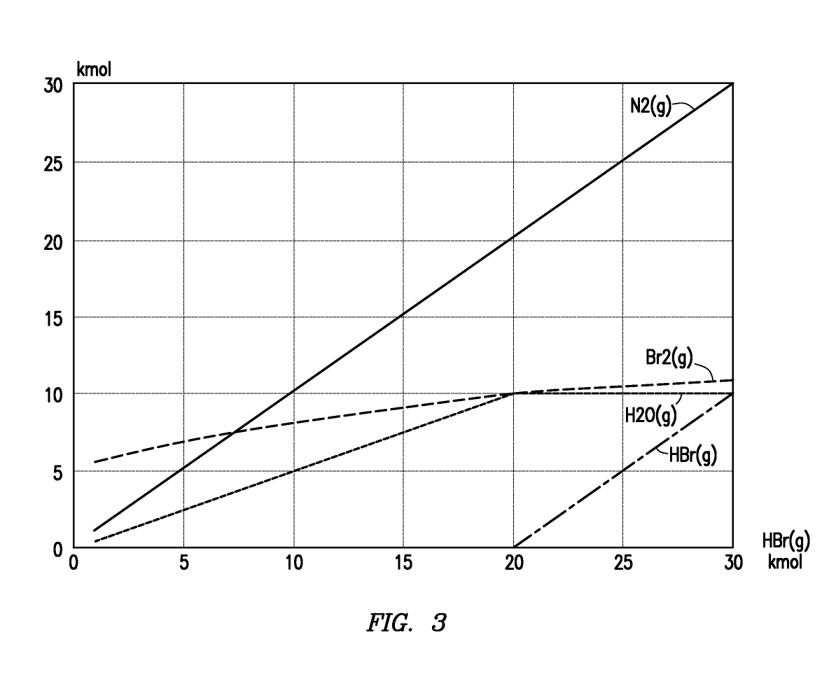

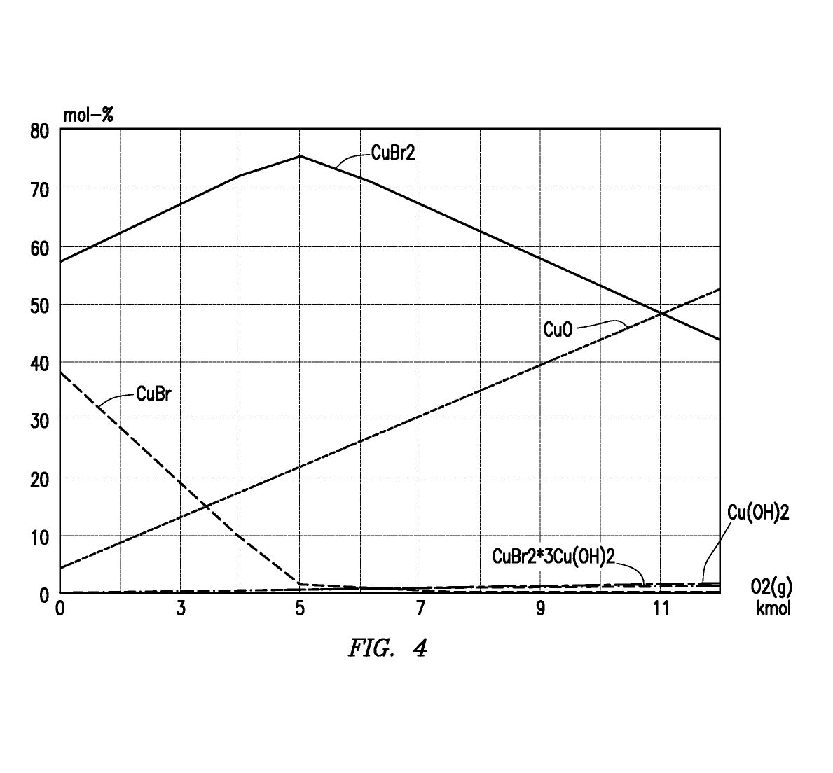

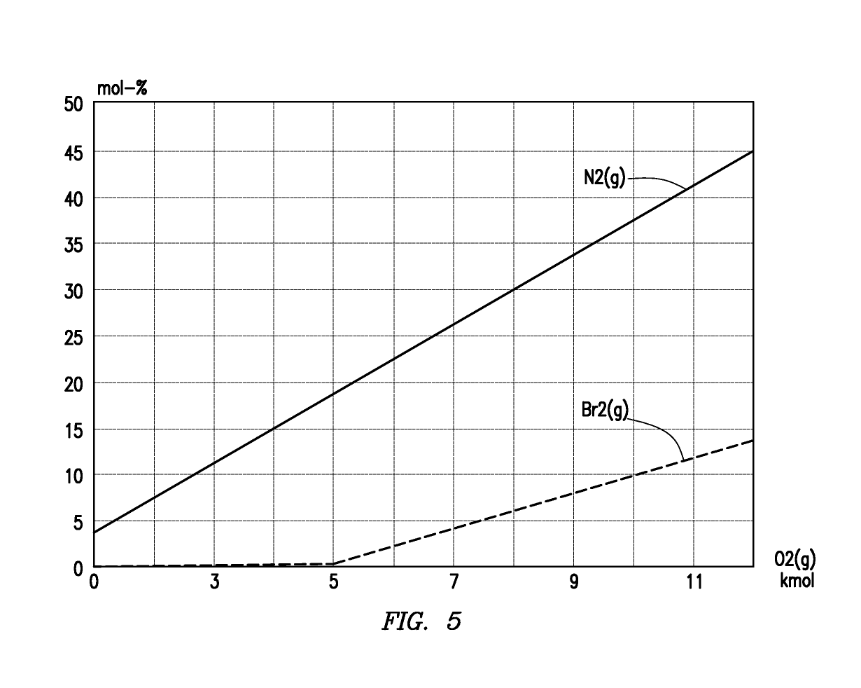

Table 2 and FIG. 2 through FIG. 5 give an example of calculated equilibrium vapor-phase and solid-phase compositions which would be expected to result from the operation of the copper oxide solid-reactant bed operated “in-line” with the process loop at a temperature of about 280° C., followed by the “off-line” regeneration with air at lower temperature of about 100° C. to restore the metal oxide content of the bed. FIGS. 2 and 4 show the equilibrium solid species versus the amount of hydrogen bromide added in the oxidation step and regeneration step, respectively. FIGS. 3 and 5 show the equilibrium vapor-phase composition versus the amount of hydrogen bromide added in the oxidation step and regeneration step, respectively. The calculated results show that the reduced metal oxide can react with molecular oxygen in the air without significant loss of bromine to the air stream and bring atomic oxygen in to the process in the form of a reactive metal oxide, useful for the oxidation of byproduct hydrogen bromide back to bromine in the bromine-based gas-conversion process.

TABLE 2
Cu System
Thermodynamic Equilibrium
Calculation

| | Mol Balance | | | | | |
|---|---|---|---|---|---|---|
| | | Step 1 | | | Step 2 | |
| | | HBr | | | CuBr | |
| | In | Oxid | Out | In | Oxid | Out |
| Gas (inert) | 20.000 | | 20.000 | 18.810 | | 18.810 |
| HBr(g) | 20.000 | | 0.000 | 0.000 | | 0.000 |
| H2O(g) | | | 10.000 | 1.190 | | 0.020 |
| O2(g) | | | | 5.000 | | 0.000 |
| Br2(g) | | | 10.000 | 0.000 | | 0.359 |
| CuO | 10.000 | | 0.000 | 0.000 | | 8.870 |
| CuBr | 0.000 | | 19.900 | 19.900 | | 0.618 |
| CuBr2 | 32.000 | 280 C. | 22.100 | 22.100 | 100 C. | 31.100 |
| CuBr2*4H2O | 0.000 | 5 bar | 0.000 | 0.000 | 5 bar | 0.000 |
| CuBr2*3Cu(OH)2 | 0.000 | | 0.000 | 0.000 | | 0.271 |
| Cu(OH)2 | 0.000 | | 0.000 | 0.000 | | 0.323 |
| Cu2O | 0.000 | | 0.000 | 0.000 | | 0.000 |

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope and spirit of the present invention. While processes and systems are described in terms of “comprising,” “containing,” or “including” various components or steps, the processes and systems can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to approximately b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an”, as used in the claims, are defined herein to mean one or more than one of the element that it introduces. If there is any conflict in the usages of a word or term in this specification and one or more patent or other documents that may be incorporated herein by reference, the definitions that are consistent with this specification should be adopted.

What is claimed is:

1. A process for producing elemental halogen comprising:
providing a first stream comprising a hydrogen halide;
contacting the first stream with a metal oxide to form water, elemental halogen, and at least some metal halide, wherein the metal oxide comprises a metal capable of forming a plurality of stable oxidation states; and
contacting the metal halide with an oxygen source to produce a regenerated metal oxide, wherein the oxygen source contacts the metal halide under conditions sufficient to avoid release of elemental halogen.

2. The process of claim 1, wherein the elemental halogen is bromine, and wherein the first stream further comprises methane.

3. The process of claim 1, wherein the metal of the metal oxide comprises at least one metal selected from the group consisting of chromium, iron, copper, tin, and vanadium.

4. The process of claim 1, further comprising contacting the water with a stripping gas to recover a fraction of the elemental halogen from the water.

5. The process of claim 4, wherein the stripping gas comprises oxygen, and the stripping gas with the recovered elemental halogen is the oxygen source.

6. The process of claim 1, wherein metal of the metal oxide comprises copper, and wherein the step of contacting the metal halide occurs at a temperature from about 25° C. to about 150° C., and wherein the step of contacting the first stream occurs at a temperature of about 150° C. to about 350° C.

7. A process for producing higher hydrocarbons comprising:
providing a first stream comprising lower molecular weight alkanes and a hydrogen halide;
contacting the first stream with a metal oxide to form oxidation products comprising water, elemental halogen, and a metal halide;
reacting at least a portion the methane and at least a portion of the elemental halogen to form halogenation products comprising alky halides and a first portion of produced hydrogen halide; and
contacting at least a portion of the alkyl halides with a catalyst to produce synthesis products comprising higher hydrocarbons and a second portion of produced hydrogen halide.

8. The process of claim 7 wherein the metal oxide comprises a metal capable of forming a plurality of stable oxidation states.

9. The process of claim 7 wherein the metal of the metal oxide comprises at least one metal selected from the group consisting of chromium, iron, copper, tin, and vanadium.

10. The process of claim 7 wherein the elemental halogen comprises bromine.

11. The process of claim 7 further comprising contacting the water with a stripping gas to recover a fraction of the elemental halogen from the water.

12. The process of claim 7 further comprising contacting the metal halide with an oxygen source to regenerate at least a portion of the produce a regenerated metal oxide.

13. The process of claim 7 wherein the catalyst comprises a zeolite catalyst.

14. The process of claim 7 further comprising combining the synthesis products with a feed gas comprising a lower molecular weight alkane and separating the combined stream into a C₁ recycle stream comprising methane and the produced alkyl halide, a C₂₋C₃ recycle stream comprising ethane and propane, and a liquid products stream comprising C₄₊ hydrocarbons.

15. The process of claim 14, wherein the methane in the C₁ recycle stream and the C₂₋C₃ recycle stream are separately brominated.

16. The process of claim 7, wherein lower molecular weight alkanes comprise methane from a feed gas.

17. The process of claim 16, wherein the feed gas comprises natural gas.

18. The process of claim 7, wherein metal of the metal oxide comprises copper, and wherein the step of contacting the first stream occurs at a temperature of about 150° C. to about 350° C., and wherein the process further comprises contacting the metal halide with an oxygen source at a temperature from about 25° C. to about 150° C.

19. A system for producing higher hydrocarbons comprising:
an oxidation unit comprising a metal oxide reactor bed for converting a hydrogen halide to a metal halide and elemental halogen, wherein at least a portion of the metal oxide in the metal oxide reactor bed is converted to a metal halide, and wherein the oxidation unit further comprises an offline metal oxide bed containing converted metal halide in fluid communication with an oxygen source;
a bromination unit in fluid communication with the oxidation unit for reacting an alkane with the elemental halogen; and
a synthesis unit in fluid communication with the bromination unit, wherein the synthesis unit comprises a catalyst for converting alkyl halides to higher hydrocarbons.

20. The system of claim 19, further comprising a separation unit in fluid communication with the synthesis unit and the oxidation unit, wherein the separation unit is configured to receive a synthesis product stream from the synthesis unit and supply a C₁ recycle stream to the oxidation unit.
